(19) United States
(12) Patent Application Publication
(10) Pub. No.: US 2015/0065955 A1
(43) Pub. Date: Mar. 5, 2015

(54) SYRINGE FOR THE INJECTION OF A MEDICAMENT WITH AUTOMATIC NEEDLE EXTENSION AND RETRACTION

(71) Applicant: Daniel Alberto Ierfino, Playa del Carmen Quintana Roo (MX)

(72) Inventor: Daniel Alberto Ierfino, Playa del Carmen Quintana Roo (MX)

(21) Appl. No.: 14/011,397

(22) Filed: Aug. 27, 2013

(51) Int. Cl. A61M 5/32

(52) U.S. Cl. CPC A61M 5/3232
USPC 604/110; 604/198

(57) ABSTRACT

A syringe for injecting medicament having automatic needle extension and retraction the syringe includes a first body with a cylindrical cross-section, which includes a front end and a rear end. The front end is provided with a means of transport smaller in diameter than the first body, which is fastened to same with a first fastening protrusion. The means of transport include a male frustoconical element which in turn includes conduits to transport said medicament, the front end further including a sealing protrusion and, located above said first fastening protrusion and said first sealing protrusion, a first alignment protrusion, inside said first body there is also a first elastic element which is fastened to said rear end of said first body with a posterior spring clip, and to said means of transport with an anterior spring clip, the rear end of said first body includes a handle.

### FIELD OF THE INVENTION

The present relates to a syringe for the injection of a medicament with automatic needle extension and retraction.

### BACKGROUND

Currently, various syringe-type devices exist for the injection of a medicament, with automatic extension and retraction of the needle.

Patents U.S. Pat. No. 7,645,265B2 and U.S. Pat. No. 7,635,356B2 disclose an injection device which comprises an outer housing inside of which is located a barrel for holding a medicament M with a needle at one extreme thereof, the needle being capable of moving in and out of the housing, and a plunger or piston moveable inside said barrel, an inner housing intermediate the outer housing and the barrel and plunger, and an energy source in communication with said inner housing which causes the same to move between a first position where the needle is exposed outside the outer housing, a second position in which the medicament M is injected through the needle, and a third position in which the needle is retracted into the outer housing.

Patent U.S. Pat. No. 7,776,015B2 discloses a jet injector device which includes a chamber for the medicament M, with a needle having an injection end which extends from said chamber to be inserted into a patient using a force-generating source configured to apply a specific pressure to the chamber to expel the medicament M through the tip of the needle.

Patent EP1569708B1 discloses a catheter having a retractable needle and a tubular plunger supported by a structure in a preferred position before and during catheter insertion. Following the insertion of the same, the plunger is pushed permitting a compressed spring to force the needle upward into the plunger.

Publication EP2588174A1 discloses a medical device having a barrel, a retractable needle, a needle retraction assembly and a plunger, said assembly held inside the barrel prior to retraction and including at least in part a retainer member which is in contact with the barrel. The plunger comprises a seal and an elastomeric web which seals a retraction cavity inside the plunger prior to the retraction of the needle. The retainer member and the plunger seal each cooperate with an inside wall of the barrel to provide a sealed liquid containment chamber within the device.

Publication EP2349414A1 discloses a pre-filled syringe having a retractable needle with a variable volume liquid containment chamber defined by surfaces made of glass or an elastomeric material, but not plastic.

Patent U.S. Pat. No. 6,391,003B1 discloses a needle-assisted jet injector having a locking mechanism to reduce the probability of contact with the needle or the re-use of the same. When the force to expel the needle has been generated, one part of the needle extends beyond the injector nozzle and penetrates the outer layer of the patient's skin to deliver the medicament via jet injection to a deeper region. After the injection, the needle is retracted into the injector nozzle.

Publication U.S.2011/0015577A1 discloses a needle assembly which is used in combination with a storage device which comprises a barrel which defines a chamber which receives the medicament M and a piston, comprising an assembly one part of which supports the needle made of a first material said needle capable of moving between an exposed and concealed position, connecting elements to secure said needle-supporting part to said barrel in its exposed position.

Patent CN201481922U discloses a disposable injector having a replaceable needle head, which can be retracted and concealed after injection via an automatic retraction mechanism, which prevents the needle from being re-used.

Patent EP1567212B1 discloses a single-use retractable syringe which comprises a handle section and a needle retention section joined to said handle section which acts as a plunger and which further serves to activate the retraction mechanism when said handle section is extended beyond the injection position. The locking mechanism in the syringe barrel permits a rearward motion of the handle to fill the chamber with medicament, and a forward motion of the handle to empty said chamber, limiting the backward motion of the needle retention chamber and/or of the handle to prevent the syringe from being re-used even if the operator were to forget to activate the needle retraction device.

Patent U.S. Pat. No. 7,993,307B2 discloses a syringe having automatic and gradual retraction of the needle which includes a plunger inside a hollow barrel, a piston in the head of the plunger and a hub at the head of the barrel. The hub includes an elastic connecting hub which connects to the hub, and which can be disconnected from the head of the barrel and retracted by the action of a spring after the injection has been completed, so that the needle is retracted into the barrel.

Publication numbers EP2438943A1, EP2438942A1, EP2438947A1, AR083348A1, AR083345A1 and AR083346A1, disclose automatic injectors for administering medicaments, which comprise a tubular chassis and a carrier sub-assembly including a tubular carrier capable of displacement within the chassis. The carrier comprises a syringe, a drive spring and a plunger to positively load the spring when it moves forward until it reaches a stop element set into the syringe. A control spring is connected to the carrier by first locking devices for inserting the needle. Second locking devices are available in to release the drive spring when the carrier reaches the position for administering the medicament. The first locking devices disconnect the control spring from the carrier, and connect it to the chassis so that it will advance over the needle thus successfully concealing the same.

Document AR0010255A1 discloses a combined retractable needle and syringe in which the needle is mounted on a retractable element which retracts into the body of the syringe plunger. Said retractable element is driven by a spring, and is activated by releasing the front end of the retractable element from its coupling with a mount located at the front end of the inside barrel of the syringe. Said front end is only released when the mount is pushed forward, extended and is detached from the retraction element of the needle.

Publication AR003355A1 discloses a needle triggering device using a plastic element which when released by said triggering device, pushes the needle against the patient's skin.

Publication AR247486A1 discloses a syringe having an auto-disable needle which is lodged in a sheath with elastic means which permit its complete retraction into said sheath.

Patent AR236442 discloses a syringe which comprises a cylinder containing a plunger with a piston, said cylinder having mounted on it a sliding tubular protector which covers the entire length of the needle, including means for defining the position of said needle between at least two positions, one outside the protector and the other disabled within the same.

Publication AR006066A1 discloses a single-use, disposable syringe having an automatic safety mechanism, which is based on the structure of two cylinders and one plunger, all made of elastomeric or plastic material. The first cylinder is open at one of its extremes, covers the needle inside it and contains a spring, and the other extreme thereof has an aperture through which the needle exits at the moment of the injection. The second cylinder, smaller in diameter, terminates in a wall having a frustoconical protrusion and a central aperture which carries the needle and is also open at its other extreme, being able to slide longitudinally within the first cylinder. The plunger has a rubber piston at one of its extremes and slides through the inside of the second cylinder and the other extreme has a groove. Both cylinders and the plunger have cylindrical or ellipsoidal winglets for manipulating the syringe. The winglets have ratchet teeth or impediments on their surfaces which constitute the automatic safety mechanism. The second cylinder is inserted into the first cylinder, and because of the effect of the impediments, they become one single unit, permitting the needle to exit. When the plunger is pulled rearward inside the second cylinder, it is filled with the liquid which will be injected into the patient and the injection will occur by reversing the action. Once the injection has been completed, the safety mechanism definitively locks the plunger in the second cylinder, and the first cylinder returns to its position when the retaining ratchet is triggered once again covering the needle.

Publication AR011360A1, by the same Applicant, discloses a syringe having an automatically retractable needle, which comprises a drive piston, a containment cylinder with a needle-carrying head, and a needle with a coupling ring.

All of the devices presented above include a cylinder and a plunger, or two cylinders and a plunger, complicated to manufacture and manipulate, and do not have a complete sealing off mechanism. In this regard, the present invention simplifies the structure and the manipulation of this type of syringe by means of the inclusion of three coaxial cylinders where the third cylinder replaces said plunger, and includes a mechanism for automatic needle extension and retraction, completely sealing the path of the medicament.

### SUMMARY

Thus the object of the present invention is a syringe for injecting medicament, having automatic extension and retraction of the needle, which comprises:

a first body with a cylindrical cross-section, which comprises a front end and a rear end; said front end is provided with a means of transport smaller in diameter than the first body, which is fastened to the same with a first fastening protrusion, said means of transport including a male frustoconical element which in turn includes conduits to transport said medicament, said front end further including a sealing protrusion and, located above said first fastening protrusion and said first sealing protrusion, a first alignment protrusion, inside said first body there is also a first elastic element which is fastened to said rear end of said first body with a posterior spring clip, and to said means of transport with an anterior spring clip, the rear end of said first body includes a handle;

a second body which has an front end and a rear end, this latter being open to allow said first body to be inserted, and provided with a stop element located inside the second body, said second body having at some point of its outer surface, a second fastening protrusion, said front end further including a second alignment protrusion, from the front end of said second body a frustoconical cavity projects which acts as a detent for the needle, from which a cylindrical space is provided concentrically, including grooves; and

a third body which further includes an front end and a rear end, open to permit the insertion of said second body, inside of which there is a flexible stop included which acts as an impediment to said second fastening protrusion of said second body, a release button being set above said flexible stop consisting of an anterior part at a right angle penetrating into said third body, and a posterior part linked to a second elastic element, said front end includes a third elastic element which is fastened to said third body via fasteners in the center of which there is an aperture whose diameter is approximately the same as that of the needle.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 shows a cross-section of the elements of the syringe according to the invention.

FIG. 2 shows a cross-section of the syringe according to the invention prior to the suction of the medicament M.

FIG. 3 shows a cross-section of the syringe according to the invention with the medicament M inside the second element.

FIG. 4 shows a cross-section of the syringe in a position prior to the injection of the medicament, with the needle concealed.

FIG. 5 shows a cross-section of the syringe according to the invention after the patient's skin has been punctured and at the point of injecting the medicament M.

FIG. 6 shows a cross-section of the syringe according to the invention during the injection of the medicament M.

FIG. 7 shows a cross-section of the syringe according to the invention after the injection of the medicament M and the retraction of the needle.

FIG. 8 shows an enlarged section defined in FIG. 2 while the needle according to the invention is in its original position.

FIG. 9 shows an enlarged section defined in FIG. 3, during the process of taking the medicament M.

FIG. 10 shows an enlarged section defined in FIG. 4 during the retraction of the needle.

FIG. 11 shows a first embodiment of the transport conduits for the medicament.

FIG. 12 shows a second embodiment of the transport conduits for the medicament.

FIG. 13 shows the moment of injection of the remaining medicament M once the transport conduits have been sealed.

### DETAILED DESCRIPTION OF THE PREFERRED EMBODIMENTS

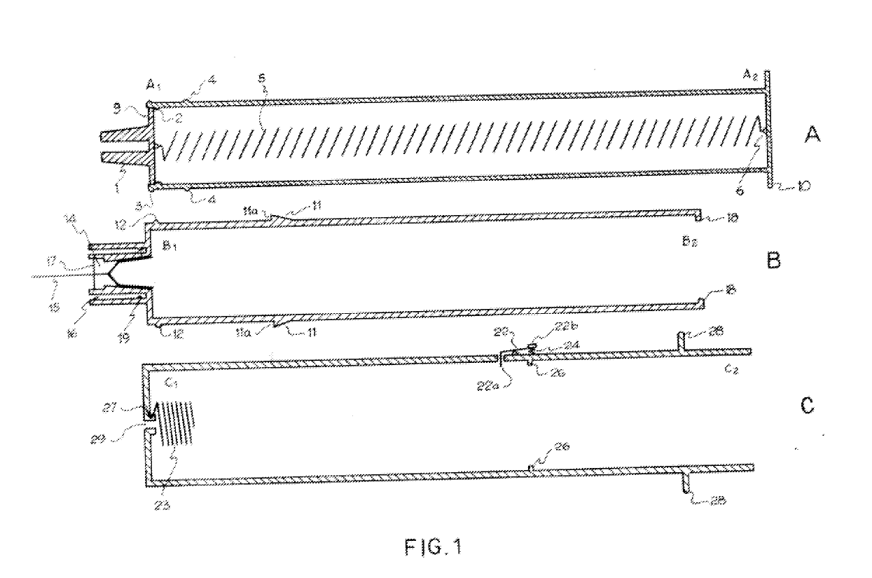

FIG. 1 shows a cross-section of the elements of the syringe according to the invention, where a first body A can be seen in cylindrical cross-section, which comprises an front end A1 and a rear end A2. Said front end A1 has a means of transport 9 of a lesser diameter than the first body A, which is fastened to the same by a first fastening protrusion 2. Said means of transport 9 includes a male frustoconical element 1 capable of connecting under pressure with the female element of a needle 15 which will be located in a second cylinder B, as we will see below. Said front end A1 also has a sealing protrusion 3 to prevent the medicament M from flowing upward.

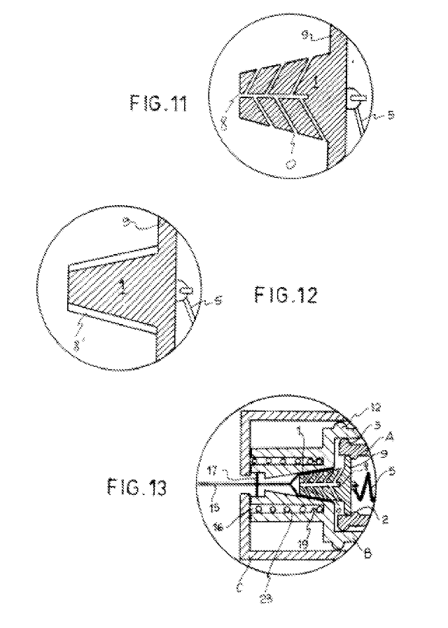

Above said first fastening protrusion 2 and said sealing protrusion 3, there is located a first alignment protrusion 4 which makes it possible to keep in the displacement of said first body A aligned inside said second body B as we will see below. Inside said first body A there is a first elastic element 5, or spring, which is fastened to the rear end A2 of the first body A with a posterior spring clip 6, and to said transport medium 9 with an anterior spring clip 7. The rear end A2 of said first body A has a handle 10 whose diameter is greater than that of said first body A, and an ellipsoidal or circular configuration, suitable for being held by the operator during the loading of the medicament M. Said male frustoconical element 1 has, in turn, transport conduits for said medicament M. Said transport conduits can be configured with circular perforations O which go from the surface of said male frustoconical element 1 to a central longitudinal conduit 8 fabricated inside said male frustoconical element, such as shown in FIG. 11, for the longitudinal conduits 8′ made along the entire outside surface of said male frustoconical element 1, as shown in FIG. 12, or for any other kind of conduit within the scope of any expert with ordinary skill in the art.

Said first body A is placed within a second body B, which also has an front end B1 and a rear end B2, the latter being open to allow for the first body A to be inserted but with the presence of a stop element 18 located inside said second body B. Said stop element 18 comes into contact with said first alignment protrusion 4 of said first body A when it is being displaced rearward, the which permits the rearward displacement of said second body B. Said second body B includes at some point of its external surface a second fastening protrusion 11 which can take the form of a right-angled triangle or step, with its short side 11a being oriented towards said front end B1, forming a right angle with the external surface of said second body B. Said front end B1 moreover, includes, a second alignment protrusion 12 which will keep the displacement of said second body B aligned inside said third body C as we will see below. From the front end of B1 of said body B, a frustoconical cavity is projected which acts as a detent for a needle 15, which prevents said needle from being displaced when it is triggered towards the body of the patient and when the ampoule which contains the medicament M is punctured, holding back said needle 15 until it receives sufficient traction force to remove it from said cavity 14.

Concentrically and outward from said frustoconical cavity 14, there is a cylindrical space 16, which includes grooves 19 for adjusting half a turn a third elastic element 23, or spring, present in a third body C as we will see below. Additionally, the anterior portion of the frustoconical cavity 14, includes a membrane 17 which is penetrated by the needle 15, to prevent the escape of any liquid.

Said second body B is inserted into a third body C which also includes an front end C1 and a rear end C2, open to permit the insertion of said second body B. From that second rear end, there are winglets 28 projected radially outward to be grasped by the operator and included within it is a flexible stop element 26 which hinders the passage of the second fastening protrusion 11 of said second body B, indicating to the operator that he/she must stop with the withdrawal of said second body B, and at the same time permitting the operator, when the injection is finished, to withdraw said second body B from said third body C, hence its flexible characteristic. Above said flexible stop element 26, a release button 22 is placed consisting of an anterior part 22a at a right angle which penetrates into said third body C, and a posterior part 22b linked to a second elastic element 24.

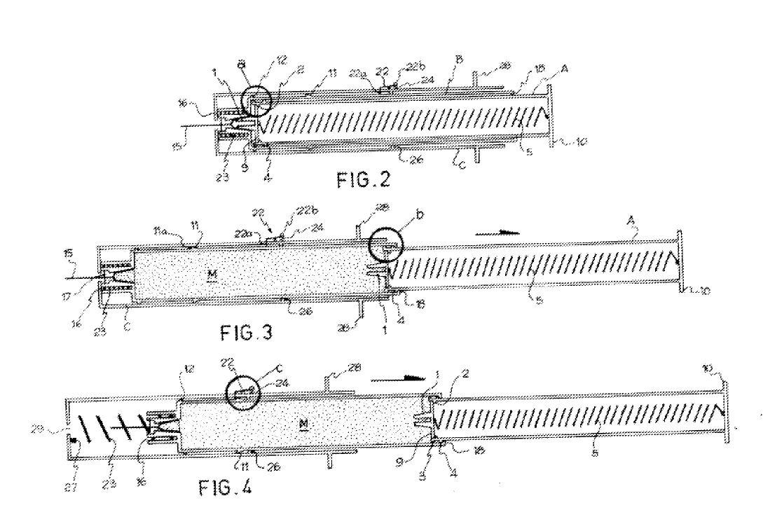

When said second fastening protrusion 11 passes under said release button 22 during the rearward displacement of said second body B, said second elastic element 24 is compressed until it strikes said flexible stop element 26, and said anterior part 22a of said release button 22 drops down in front of said base 11a of said second fastening protrusion 11 as a consequence of the pressure exercised by the decompression of said second elastic element 24, locking the mechanism and readying the needle for insertion into the body of the patient. In said front end C1 is a said third elastic element 23, which is adjusted half a turn within said cylindrical space 16 via said grooves 19 in said second body B. Said third elastic element 23 is fastened to said third body C with fasteners 27 which have an aperture 29 in the center, approximately equal in size to the diameter of the needle 15 so that it can pass through the same. Said third body C can have a toy format and include colors if the patient is a child, so that the injection will not be traumatic. When the injection is completed, said third body C can be given to the child. The syringe assembly with the three bodies A, B and C can be seen in FIG. 2, where an enlarged area in a circle shows the interrelation of parts detailed in FIG. 8.

Said first body A serves as a plunger and as a means for automatic retraction of the needle 15. Said second body B contains the medicament M and will retain said needle 15. Said third body C permits inserting the needle into the patient, and can be separated from said second body B after the injection has been completed.

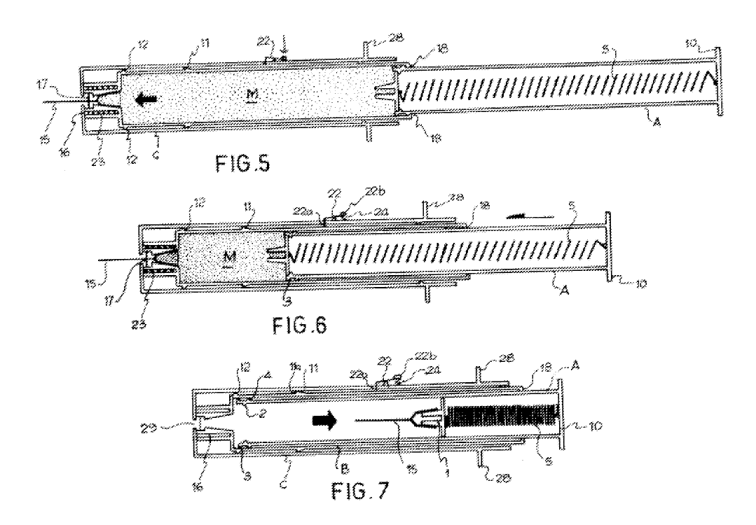

The operation of the syringe has 5 principal stages:
1. to load the medicament M (see FIG. 3);
2. to conceal the needle (see FIG. 4);
3. to insert the needle into the patient (see FIG. 5);
4. to inject the medicament M (see FIG. 6);
5. to retract the needle (see FIG. 7); and
6. to give the body C to the patient (this step is optional and not shown).

The medicament M is loaded in the traditional way by pulling rearward on said first body A, as shown in FIG. 3, where an enlarged area in a circle shows the interrelation of parts detailed in FIG. 9. Even if the medicament M has already entered said second body B, the operator continues to pull on said first body A until said release button 22 retains said second body B automatically within the third body C, as mentioned above. When the operator pulls rearward on said first body A, said second body B will also be displaced and both will encounter the stop elements. Specifically, said first body A is braked by said first alignment protrusion 4 in said stop element 18 of the body B, and said second fastening protrusion 11 of said second body B encounters said flexible stop element 26 of said third body C.

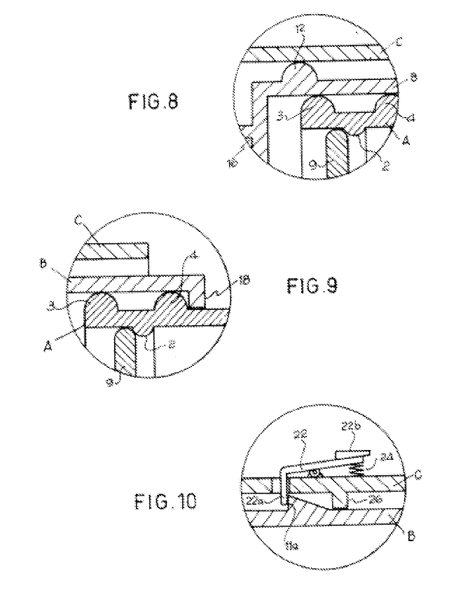

As mentioned above, said anterior part 22a of said release button 22 drops down in front of said base 11a of said second fastening protrusion 11 as a consequence of the pressure exercised in a downward direction by said third elastic element 24, locking the second body B, which is then ready to inject as shown in FIG. 4, where an enlarged area in a circle shows the interrelation of parts detailed in FIG. 10.

The operator rests the anterior part of the syringe on the patient to proceed with the injection, at that moment the operator presses the release button to release said second body B, driving said needle 15 into the body of the patient, as shown in FIG. 5. Subsequently, the operator will inject the medicament M by pushing said first body A to the end, as shown in FIG. 6, and, with a slight additional pressure, said male frustoconical element will mate with said frustoconical cavity 14. The transport conduits for said medicament M will permit the passage of the same, even after it has been sealed, however, some space will still remain to advance and complete the mating and adjust said male element 1 in said needle 15. In other words, the medicament M remaining in body B, will pass via said transport conduits, reaching said needle 15 to be able to be injected into the patient as shown in FIG. 13.

On the one hand, the retraction of said needle 15 is achieved by a combination of forces, the mating of the male element 1 in frustoconical cavity 15 and the release of the means of transport 9. The needle 15 will be released when the male element 1 captures it, and the transport means 9 released from the first fastening protrusion 2, as shown in FIG. 7. As a consequence, said first elastic means 5 will begin to exert a much greater retractive force than the fastening force of said frustoconical cavity 14. The frustoconical cavity 14 has a double function with respect to the needle 15.

On the other hand, it holds the needle 15 so that it will not be dislodged from its place until it receives a specific force of traction, and on the other hand, its concave shape will not permit the needle 15 to move outward when the male element 1 enters into the needle 15 and exerts force on it until entering into the cavity under pressure.

Both in the case when the operator discards the syringe with the three bodies and also if he/she decides to give the third body C to a child, there are no risks of contamination either for the operator or for the child since the second body B is completely sealed making punctures or the exit of fluids impossible, and the third body C which the child gets is free of contaminants.

For transport and/or manipulation, the syringe includes a small plastic element (not shown) under the release button 22, to prevent said release button 22 from being pressed accidentally. Said small plastic element can be easily removed when the operator deems it advisable. Another way of transporting the syringe is in a protective sleeve for the needle 15 (not shown), for which the anterior part C1 of said third body C must include a corresponding fastening protrusion to fasten it to said protective sleeve for the needle 15.

It is understood, therefore, that this invention is not limited to the particular embodiments disclosed, but is intended to cover all modifications which are within the spirit and scope of the invention as defined by the appended claims; the above description; and/or shown in the attached drawings.

1. A syringe for injecting a medicament (M) having automatic needle extension and retraction, the syringe comprises:
a first body (A) with a cylindrical cross-section, which comprises an front end (A1) and a rear end (A2); said front end (A1) is provided with a means of transport (9) smaller in diameter than the first body (A), which is fastened to same with a first fastening protrusion (2), said means of transport (9) including a male frustoconical element (1) which in turn includes conduits to transport said medicament (M), said front end (A1) further including a sealing protrusion (3) and, located above said first fastening protrusion (2) and said first sealing protrusion (3), a first alignment protrusion (4), inside said first body (A) there is also a first elastic element (5) which is fastened to said rear end (A2) of said first body (A) with a posterior spring clip (6), and to said means of transport (9) with an anterior spring clip (7), the rear end (A2) of said first body (A) includes a handle (10);
a second body (B) which has an front end (B1) and a rear end (B2), this latter being open to allow said first body (A) to be inserted and provided with a stop element (18) located inside the second body (B), said second body (B) moreover having at some point of its external surface second fastening protrusion (11), including on said front end (B1) also a second alignment protrusion (12), from the front end (B1) of said second body (B) a frustoconical cavity projects (14) which acts as a detent for the needle (15), from which a cylindrical space is provided concentrically (16) including grooves (19); and a third body (C) which further includes an front end (C1) and a rear end (C2), open to permit the insertion of said second body (B), a flexible stop element being included in its interior (26) which acts as an impediment to said second fastening protrusion (11) of said second body (B), there being located above said flexible stop element (26) a release button (22) consisting of an anterior part (22a) at a right angle penetrating into said third body (C), and a posterior part (22b) linked to a second elastic element (24), said front end (C1) includes a third elastic element (23) which is fastened to said third body (C) via fasteners (27) in the center of which there is an aperture (29) whose diameter is approximately the same at that of the needle (15).

2. A syringe according to claim 1, wherein said male frustoconical element (1) is capable of connecting under pressure with said frustoconical cavity (14).

3. A syringe according to claim 1, wherein said first alignment protrusion (4) allows a displacement of said first body (A) to be kept aligned inside said second body (B).

4. A syringe according to claim 1, wherein said first stop element (18) comes into contact with the first alignment protrusion (4) of said first body (A) when it is being displaced rearward, which permits the rearward displacement of said second body (B).

5. A syringe according to claim 1, wherein said second alignment protrusion (12) allows displacement of said first body (B) to be kept aligned inside said third body (C).

6. A syringe according to claim 1, wherein said grooves (19) adjust by half a turn the third elastic element (23) present in said third body (C).

7. A syringe according to claim 1, wherein said flexible stop element (26) inhibits the passage of said second fastening protrusion (11) of said second body (B).

8. A syringe according to claim 7, wherein said second fastening protrusion (11) is formed as a right-angled triangle or step, with a short side (11a) thereof being oriented towards the front end (B1) forming a right angle with the external surface of said second body (B).

9. A syringe according to claim 1, wherein said second fastening protrusion (11) passes under said release button (22) during rearward displacement of said second body (B), said second elastic element (24) is compressed until said second fastening protrusion (11) strikes said flexible stop element (26), and said anterior part (22a) of said release button (22) drops down in front of said base (11a) of said second fastening protrusion (11) as a consequence of the pressure exercised by the decompression of said second elastic element (24),

10. A syringe according to claim 1, wherein said first body (A) acts as a plunger and as a means of automatic retraction of said needle (15), said second body (B) contains a medicament (M) and retains said needle (15) and said third body (C) allows the needle to be released for insertion (15) into the patient, and can be separated from said second body (B) after the injection has been completed.

11. A syringe according to claim 1, wherein said handle (10) has a diameter greater than that of said first body (A) and has an ellipsoidal or circular configuration.

12. A syringe according to claim 1, wherein from said rear end (C2) of said third body (C) winglets (28) project radially outward.

13. A syringe according to claim 1, wherein said third body (C) is formed as a toy and includes colors, so that once the injection is completed, said third body (C) can be given to a child.

14. A syringe according to claim 1, wherein the syringe includes under the release button (22), a small plastic element to prevent the release button (22) from being pressed accidentally, which can be easily removed when the operator deems it advisable.

15. A syringe according to claim 1, wherein the syringe includes a protective sleeve for said needle (15), for which the anterior part C1 of said third body C has a corresponding fastening protrusion to fasten it to said protective sleeve for said needle (15).

16. A syringe according to claim 1, wherein the anterior part of said frustoconical cavity (14), includes a membrane (17), to prevent the escape of any liquid, which is penetrated by said needle (15).

17. A syringe according to claim 1, wherein said transport conduits are composed of circular perforations (O) which go from the surface of said male frustoconical element (1) to a central longitudinal conduit (8) fabricated inside said male frustoconical element (1).

18. A syringe according to claim 1, where said transport conduits are composed of longitudinal conduits (8′) made along the entire external surface of said male frustoconical element (1).
